(19) United States
(12) Patent Application Publication
(10) Pub. No.: US 2025/0132024 A1
(43) Pub. Date: Apr. 24, 2025

(54) MEDICAL INFORMATION PROCESSING APPARATUS

(71) Applicant: CANON MEDICAL SYSTEMS CORPORATION, Otawara-shi (JP)

(72) Inventor: Shota NEGISHI, Otawara (JP)

(73) Assignee: CANON MEDICAL SYSTEMS CORPORATION, Otawara-shi (JP)

(21) Appl. No.: 18/921,216

(22) Filed: Oct. 21, 2024

(30) Foreign Application Priority Data
Oct. 24, 2023 (JP) 2023-182240

(51) Int. Cl. G16H 40/20; G16H 70/20

(52) U.S. Cl. CPC G16H 40/20; G16H 70/20

(57) ABSTRACT

A medical information processing apparatus according to the present disclosure includes a display and processing circuitry. The processing circuitry is configured to identify a scene in a medical treatment performed on a patient as an identified scene, calculate attribute information relating to an attribute of the identified scene, search a medical database to acquire a piece of reference data that matches the identified scene based on the attribute information, wherein medical data generated in the past is accumulated in the medical database, and cause the display to display the piece of the reference data.

### CROSS REFERENCE TO RELATED APPLICATIONS

This application is based upon and claims the benefit of priority from the prior Japanese Patent Application No. 2023-182240, filed on Oct. 24, 2023, the entire contents of which are incorporated herein by reference.

### FIELD

Embodiments disclosed herein and the accompanying drawings relate to medical information processing apparatus.

### BACKGROUND

There are differences in experience among medical technicians who perform medical examinations or medical treatments in medical sites. In order to compensate for the lack of experience, it often happens that during a medical treatment performed on a patient, an experienced medical technician stands next to a medical technician who does not have enough experience, or an experienced medical technician remotely provides advice to a medical technician who does not have enough experience. During medical image interpretation, even an experienced doctor may sometimes refer to medical images of similar cases or relating data of the past to finally reach a diagnosis.

When dealing with an examination apparatus, a clinical examination technician often sets parameters of the examination apparatus based on the past experiences. In order to provide various medical treatments with a certain level of quality, it is expected that a medical information processing apparatus that provides a support to compensate for the lack of experience be provided.

### BRIEF DESCRIPTION OF THE DRAWINGS

FIG. 1 is a block diagram showing an example for explaining the entire configuration of a medical information processing system according to a first embodiment.

FIG. 2 is a block diagram showing an example of an internal configuration of a medical information processing apparatus included in the medical information processing system shown in FIG. 1.

FIG. 3 is a diagram for explaining the relationship among medical procedure record data, medical treatments, scenes, and a medical database in the medical information processing system shown in FIG. 1.

FIG. 4 shows an example of a data structure of medical data stored in a medical database constructed in a medical data memory included in the medical information processing system shown in FIG. 1.

FIG. 5 is a flowchart used for explaining a medical information generation process performed by the medical information processing apparatus shown in FIG. 1.

FIG. 6 is a diagram for explaining a specific example of the contents of the medical information generation process when a catheter treatment is performed on a patient as a medical treatment.

FIG. 7 shows an example of the structure of a medical data management table used for managing the medical data stored in the medical data memory included in the medical information processing system shown in FIG. 1.

FIG. 8 shows an example of a proficiency score calculation table used by the medical information processing apparatus to calculate a proficiency score in the embodiment.

FIG. 9 is a diagram for explaining an example of a logic with which the medical information processing apparatus calculates a favorable prognosis score in the embodiment.

FIG. 10 is a flowchart used for explaining a medical information search process performed by the medical information processing apparatus shown in FIG. 1.

FIG. 11 is a diagram for explaining the flow of data (on the patient's side where a medical treatment is performed) in the medical information search process when a catheter treatment is performed as the medical treatment in the embodiment.

FIG. 12 is a diagram for explaining the flow of data (accumulated in the medical database in the past) in the medical information search process when a catheter treatment is performed as the medical treatment in the embodiment.

### DETAILED DESCRIPTION

Embodiments of a medical information processing apparatus and a medical information processing system including the medical information processing apparatus will now be described with reference to the accompanying drawings. In the following descriptions, elements having substantially the same structure and functions have the same numerical symbols, and the explanation of such elements is repeated only when it is necessary to do so.

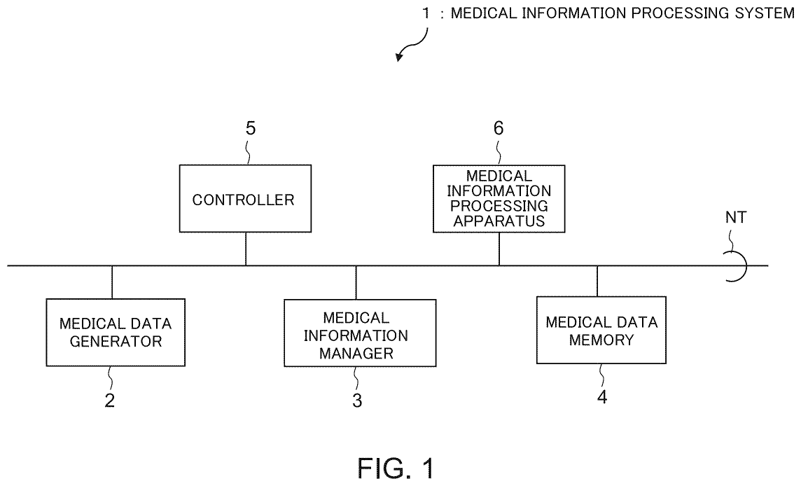

FIG. 1 is a block diagram for explaining the entire configuration of a medical information processing system 1 according to an embodiment. As shown in FIG. 1, the medical information processing system 1 according to the embodiment constitutes a so-called intra-hospital system, and includes a medical data generator 2, a medical information manager 3, a medical data memory 4, a controller 5, and a medical information processing apparatus 6. The medical data generator 2, the medical information manager 3, the medical data memory 4, the controller 5, and the medical information processing apparatus 6 are mutually connected via a network NT. The network NT herein is a wired network, a wireless network, of a combination of wired and wireless networks, and constitutes an intra-hospital network. If necessary, the network NT may be connected to an external network.

The medical data generator 2 includes such elements as a medical image diagnosis device, a bimetal measurement device, and a wearable device, and generates various kinds of medical data. Examples of the medical image diagnosis device include an X-ray computed tomography (CT) device, a magnetic resonance imaging

(MRI) device, an X-ray diagnosis device, and an ultrasonic diagnosis device. The medical data generator 2 receives an operation request from the controller 5 to perform scanning of a patient, for example. The medical data generator 2 then supplies the medical data generated by the scan to the network NT. For example, the supplied data is stored in the medical data memory 4 or processed by the medical information processing apparatus 6.

The medical information manager 3 includes, for example, a computer device such as a server, a work station, or a personal computer, and manages the medical data stored in the medical data memory 4. When a user inputs a certain kind of medical information through the controller 5, the medical information manager 3 let the inputted medical information be stored in association with the medical data stored in the medical data memory 4, if necessary.

The medical data memory 4 includes, for example, a high-capacity hard disk drive or a data server, and stores the medical data generated in the medical information processing system 1. The medical data memory 4 allows an access to the stored medical data from another device via the network NT and supplies the requested medical data to the other device.

For example, the controller 5 is operated by a user to operate or control various connected devices via the network NT. The controller 5 includes, for example, a terminal device such as a personal computer, a tablet, or a smartphone. The controller 5 may be a device carried by the user or set at a predetermined location. If necessary, the controller 5 may include such devices as a track ball, a switch button, a mouse device, a keyboard, a touchpad used for performing an input operation by touching an operation surface thereof, a touchscreen including a display and a touchpad that are integrally formed, contactless input circuitry including an optical sensor, and/or audio input circuitry.

The medical information processing apparatus 6 includes, for example, a computer device such as a server, a work station, or a personal computer, and acquires and processes the medical data generated by the medical data generator 2, or acquires and processes the medical data stored in the medical data memory 4. The medical information processing apparatus 6 may be constituted either by a single computer device or a plurality of computer devices. If the medical information processing apparatus 6 includes more than one computer device, each of the computer devices may have a different role, or multiple computer devices may cooperate to perform a single process.

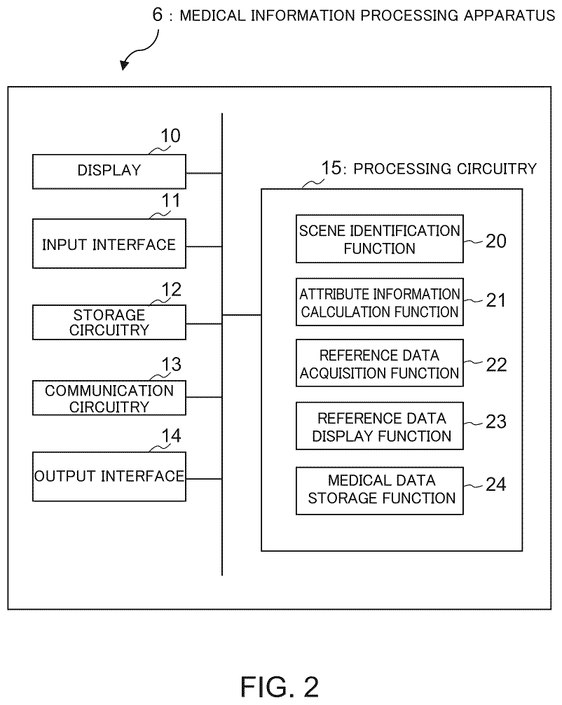

FIG. 2 is a block diagram showing an example of the configuration of the medical information processing apparatus 6 according to the embodiment. The medical information processing apparatus 6 shown in FIG. 2 includes a display 10, an input interface 11, storage circuity 12, communication circuitry 13, an output interface 14, and processing circuitry 15.

The display 10 displays various kinds of information. For example, the display 10 displays a test result of a patient's sample acquired by the medical information processing apparatus 6, the medical information of the patient generated by the medical information processing apparatus 6, or a graphical user interface (GUI) for receiving various instructions from the user. In the embodiment, the display 10 includes such an element as a liquid crystal display or an organic light emitting diode (OLED) display.

In the embodiment, the medical information processing apparatus 6 may use a display of the controller 5, through the network NT, to display various kinds of information. In this aspect, the display 10 of the medical information processing apparatus 6 may be omitted.

For example, the input interface 11 receives various inputs, converts the received inputs to electrical signals, and outputs the electrical signals to the processing circuitry 15. The input interface 11 includes, for example, a track ball, a switch button, a mouse device, a keyboard, and/or numeric keys. If the display 10 is a touch panel, the display 10 may have a function of the input interface 11.

In the embodiment, the medical information processing apparatus 6 may receive inputs from the controller 5 via the network NT. Specifically, the user's inputs received by the controller 5 are converted to electrical signals and sent as information to the communication circuitry 13 of the medical information processing apparatus 6 via the network NT. The medical information processing apparatus 6 may be controlled by the received information, which was originally the inputs by the user. In this aspect, the input interface 11 of the medical information processing apparatus 6 may be omitted.

For example, the storage circuity 12 includes a recording medium that can be read by a processor, such as a semiconductor memory element like a random-access memory (RAM) or a flash memory, a hard disk, or an optical disc. In particular, the storage circuity 12 in the embodiment stores a program used by the processing circuitry 15 and data obtained as a calculation result when the program is executed.

The medical information processing apparatus 6 in the embodiment may use the medical data memory 4 as an external memory via the network NT. Specifically, the medical information processing apparatus 6 may transmit data as medical data from the communication circuitry 13 to the medical data memory 4 via the network NT, so that the medical data may be stored in the medical data memory 4. The medical information processing apparatus 6 may also receive data as medical data at the communication circuitry 13, the data being transmitted from the medical data memory 4 via the network NT.

The communication circuitry 13 provides interface functions needed for the medical information processing apparatus 6 to send and receive information via the network NT to and from the medical data generator 2, the medical data memory 4, and the controller 5. The communication circuitry 13 has information communication protocols suitable for devices to or from which information is transmitted or received via the network NT. The communication circuitry 13 follows the information communication protocols to communicate with the other devices via the network NT.

The output interface 14 outputs various kinds of information based on signals supplied from the processing circuitry 15, for example. In the embodiment, the output interface 14 may be realized as a speaker that outputs sounds or a printer that prints data.

The medical information processing apparatus 6 in the embodiment may transmit, via the network NT, to the controller 5 information to be outputted. In this case, the controller 5 provides an output to the user based on the received information to be outputted. In this aspect, the output interface 14 of the medical information processing apparatus 6 may be omitted.

The processing circuitry 15 is calculation circuitry configured to perform various kinds of calculations, and includes a processor such as a CPU or a GPU. For example, the processing circuitry 15 according to the embodiment retrieves a piece of medical data that matches a condition from the medical data memory 4, and performs various processes on the piece of medical data obtained from the medical data memory 4.

In order to do so, the processing circuitry 15 according to the embodiment has a scene identification function 20, an attribute information calculation function 21, a reference data acquisition function 22, a reference data display function 23, and a medical data storage function 24 as shown in FIG. 2. The scene identification function 20 corresponds to a scene identifier, the attribute information calculation function 21 corresponds to an attribute information calculator, the reference data acquisition function 22 corresponds to a reference data acquirer, the reference data display function 23 corresponds to a reference data display, and the medical data storage function 24 corresponds to a medical data storage in the embodiment.

In the embodiment shown in FIG. 2, respective processing functions of the scene identification function 20, the attribute information calculation function 21, the reference data acquisition function 22, the reference data display function 23, and the medical data storage function 24 are stored in the storage circuity 12 in the form of programs that are executable by a computer. The processing circuitry 15 is a processor that reads a program from the storage circuity 12 and executes the program to realize the function corresponding to the program. In other words, the processing circuitry 15 having read the respective programs has the respective functions of the processing circuitry 15 shown in FIG. 2. Although the processing circuitry 15 alone performs the scene identification function 20, the attribute information calculation function 21, the reference data acquisition function 22, the reference data display function 23, and the medical data storage function 24 in the descriptions with reference to FIG. 2, a plurality of independent processors may be combined to constitute the processing circuitry 15, and the functions may be performed by the respective processors each executing a program.

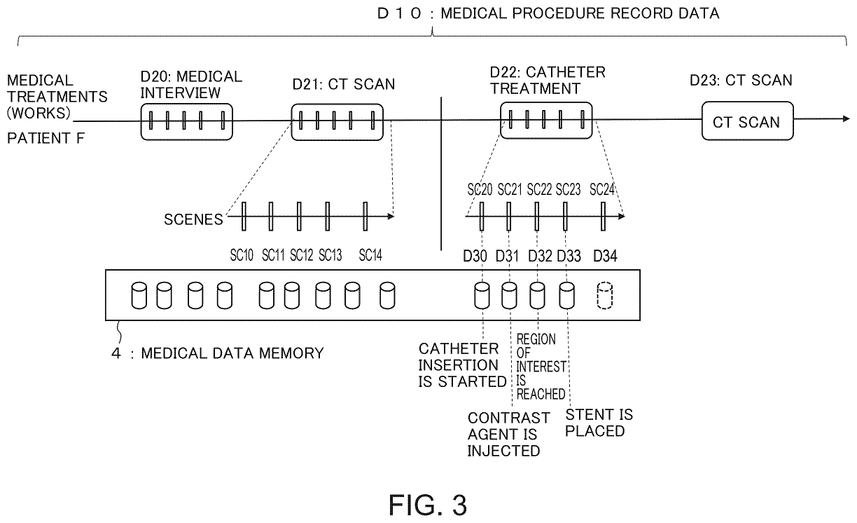

Next, the data structure of medical procedure record data D10 managed by the medical information processing system 1 according to the embodiment will be described with reference to FIG. 3. FIG. 3 is a diagram for explaining the relationship among the medical procedure record data D10, medical treatments, scenes, and a medical database. The medical database is included in the medical data memory 4 and has information corresponding to each scene as medical data.

As shown in FIG. 3, in the embodiment, the medical data is stored as the medical procedure record data D10 of each patient. Specifically, a single piece of medical procedure record data D10 may be generated and stored for each patient. The medical procedure record data D10 has a data structure associated with a pathway along the flow of time, the data structure indicating a past medical treatment performed on the patient, a current medical treatment performed on the patient, and a future medical treatment that would be performed on the patient.

In the example of FIG. 3, medical treatments performed on a patient F is shown as the medical procedure record data D10. Specifically, according to the medical procedure record data D10 shown in FIG. 3, the patient F has received medical treatments in the past, which are a medical interview D20 and a CT scan D21, is now receiving a medical treatment, which is a catheter treatment D22, and will receive a medical treatment in the future, which is a CT scan D23. In the embodiment, the medical treatments are also defined as works. Each medical treatment corresponds to one work.

One medical treatment, in other words one work, includes one or more scenes. In the example of FIG. 3, the medical treatment of the CT scan D21 includes a plurality of scenes SC10 to SC14. The medical treatment of the catheter treatment D22 includes a plurality of scenes SC20 to SC24. The number of scenes in each medical treatment may be arbitrarily determined. Generally, the more a medical treatment is complexed or the longer time it takes for performing a medical treatment, the more the number of scenes becomes.

In the specific example of the catheter treatment D22, at the scene SC20 a catheter is inserted, at the scene SC21 a contrast agent is injected, at the scene SC22 the tip of the catheter reaches a region of interest, and at the scene SC23 a stent is placed at a target point. Thus, during the catheter treatment D22, a plurality of scenes are generated along a pathway, which is a flow of time.

In the embodiment, image data taken at each scene is stored as the medical data in the medical data memory 4. For example, if the medical data generator 2 such as an X-ray imaging device is taking an X-ray image of a patient to whom the catheter is to be inserted, the X-ray image data is stored as the medical data in the medical data memory 4. In the example of FIG. 3, a piece of image data D30 that is associated with the scene SC20, a piece of image data D31 that is associated with the scene SC21, a piece of image data D32 that is associated with the scene SC22, and a piece of image data D33 that is associated with the scene SC23 are stored as the medical data in the medical data memory 4. A piece of image data D34 associated with the next scene, which is the scene SC24, will soon be stored in the medical data memory 4.

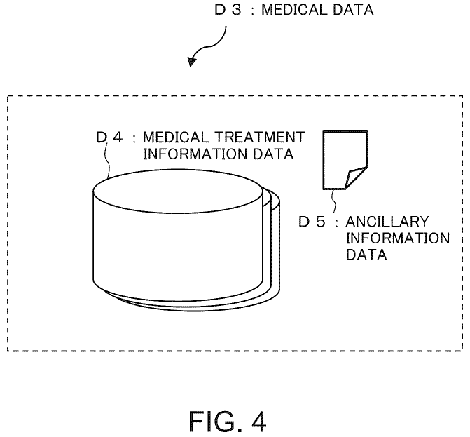

FIG. 4 shows the details of a data structure example of the medical data D3 in the medical database constituted in the medical data memory 4 according to the embodiment. As shown in FIG. 4, the medical data D3 in the embodiment includes medical treatment information data D4 of the patient associated with each scene and ancillary information data D5 associated with the medical treatment information data D4. In the example of FIG. 3, a piece of medical data D3 is generated in association with the scene SC20, and another piece of medical data D3 is generated in association with the scene SC21. Thus, a piece of medical data D3 is generated for each scene.

The medical treatment information data D4 is generated in each scene. In the example of FIG. 3, the image data obtained in each scene corresponds to the medical treatment information data D4. For example, the image data D30 obtained in the scene SC20 constitutes the medical treatment information data D4. Thus, the image data D30 is an example of the medical treatment information data D4. What kind of data corresponds to the medical treatment information data D4 depends on the type of the medical treatment, or the type of the scene. For example, the image data generated by an optical camera may correspond to the medical treatment information data D4, or each piece of setting data of the medical data generator 2 may correspond to the medical treatment information data D4.

The ancillary information data D5 is associated with the medical treatment information data D4, and has information for identifying the contents of the medical treatment information data D4. For example, the ancillary information data D5 is formed as character information, and is searched for when the medical treatment information data D4 is retrieved.

The ancillary information data D5 will be described in detail later. In the embodiment, the ancillary information data D5 is automatically generated by the medical information processing apparatus 6 while one scene in a medical treatment is being performed, and also when one scene is ended. For example, the medical information processing apparatus 6 performs an image analysis of the image data obtained from the medical data generator 2 to calculate image feature amounts such as a pattern, a width, and a narrowed state of blood vessels, and to store the image feature amounts as the ancillary information data D5.

In the embodiment, the ancillary information data D5 is also automatically generated as medical treatment summary information by the medical information processing apparatus 6 when one medical treatment is finished. The type of information stored as the ancillary information data D5 may be arbitrarily determined, and examples of the ancillary information data D5 include the type of the performed medical treatment, and the name and the degree of proficiency of a medical technician who performed the medical treatment. The medical information processing apparatus 6 may add information relating to the tendency of favorable prognosis to the ancillary information data D5 later. Examples of the information added to the ancillary information data D5 include the one-year recurrence rate, the five-year survival rate, and the rate of sequelae relating to the medical technician who have performed the medical treatment. The ancillary information data D5 thus generated is not associated with each piece of the medical data D3 but associated with the medical treatment performed on the patient.

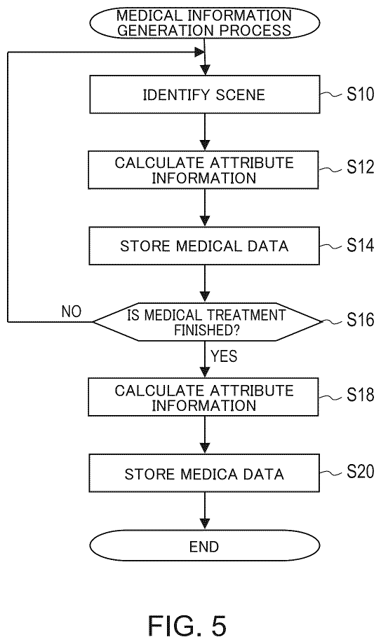

Next, a medical information generation process performed by the medical information processing apparatus 6 according to the embodiment will be described with reference to FIG. 5. The medical information generation process is executed when the processing circuitry 15 reads and performs a medical information generation process program stored in the storage circuity 12 of the medical information processing apparatus 6. The medical information generation process may start when a user such as a medical technician or a clinical examination technician inputs the starting of the medical treatment to the medical information processing apparatus 6.

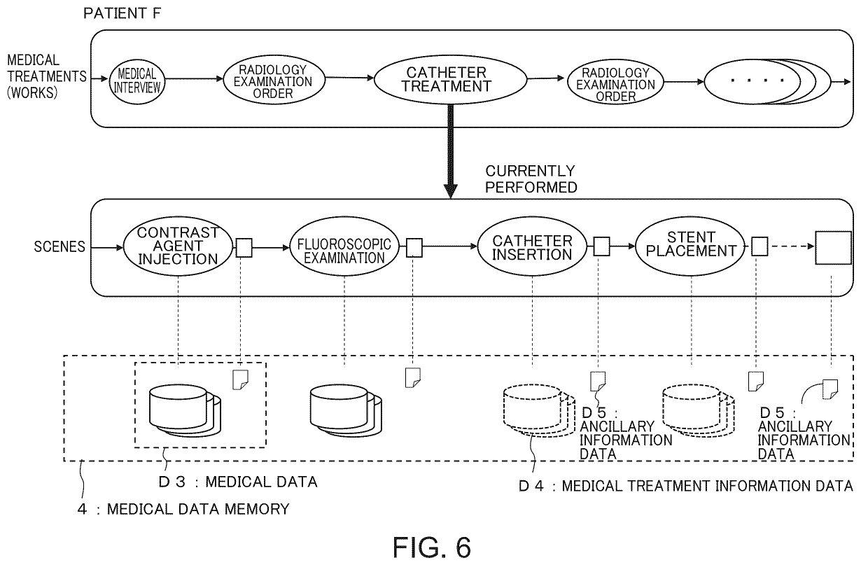

In the following descriptions, the medical information generation process shown in FIG. 5 will be explained with reference to FIG. 6, taking a catheter treatment performed on the patient F as an example of the medical treatment. Specifically, the medical information generation process will be explained by referring to the medical procedure record data D10 of a patient shown in FIG. 6. The medical information generation process is performed in the same manner for other medical treatments.

As shown in FIG. 5, in the medical information generation process according to the embodiment, the medical information processing apparatus 6 first identifies a scene in the medical treatment performed on the patient (step S10). Specifically, the scene identification function 20 of the processing circuitry 15 identifies a scene in the medical treatment performed on the patient. In the embodiment, for example, the scene identification function 20 acquires image data from the medical data generator 2 to perform image analysis of the acquired image data. Based on the result of the analysis of the image data, the scene identification function 20 identifies the scene in the medical treatment performed on the patient.

In the example of FIG. 6, the scene identification function 20 performs the image analysis and identifies the scene as the scene for inserting a catheter included in a catheter treatment, which is a medical treatment performed on the patient. When identifying the scene, the scene identification function 20 may take the sequential order of a plurality of scenes into account. For example, a scene that comes after a contrast agent injection scene and a fluoroscope examination scene may be determined as a catheter insertion scene if the treatment proceeds without a problem.

When identifying a scene, the scene identification function 20 may acquire information on the medical treatment performed on the patient in advance. For example, the scene identification function 20 may determine in advance the medical treatment to be performed on the patient based on the input to the controller 5 performed by the user. In the example of the descriptions here, the scene identification function 20 may determine in advance that the medical treatment to be performed on the patient is the catheter treatment. In the example of FIG. 6, the scene identification function 20 may determine in advance that the medical treatment performed on the patient is the catheter treatment. As a result, the degree of accuracy in identifying the scene by the scene identification function 20 may be improved.

Next, as shown in FIG. 5, the medical information processing apparatus 6 calculates attribute information relating to the attribute of the scene identified in step S10 (step S12). Specifically, the attribute information calculation function 21 of the processing circuitry 15 calculates the attribute information relating to the attribute of the scene identified by the scene identification function 20. In the example of FIG. 6, such features as the pattern, the width, and the narrowed state of a blood vessel to which a catheter is to be inserted are calculated as the attribute information. Thus, the attribute information may be calculated by performing an image analysis of the image data acquired from the medical data generator 2 by the scene identification function 20. Since a single scene in a medical treatment may have various states and situations, the attribute information in the embodiment is generated by extracting information of such various states and situations as feature amounts.

Next, as shown in FIG. 5, the medical information processing apparatus 6 has the medical data memory 4 store the medical data D3 (step S14). Specifically, the medical data storage function 24 of the processing circuitry 15 has the medical data memory 4 store the medical treatment information data D4 associated with the patient together with the attribute information calculated by the attribute information calculation function 21 as the medical data D3. In the embodiment, the attribute information corresponds to the ancillary information data D5 stored in association with the medical treatment information data D4. In the example of FIG. 6, what is stored as the medical treatment information data D4 is the image data acquired from the medical data generator 2.

Thereafter, as shown in FIG. 5, the medical information processing apparatus 6 determines whether one medical treatment is finished (step S16). Specifically, when the scene identification function 20 identifies a scene, whether all the necessary scenes have been identified and one medical treatment is finished is determined. If the medical treatment has not been finished yet (NO at step S16), the medical information processing apparatus 6 returns to step S10 and repeats the steps from the identification of the scene.

The user may send via the network NT an instruction for finishing the medical treatment to the medical information processing apparatus 6 by inputting the instruction to the controller 5. The end of the medical treatment may be inputted by the user when the medical treatment performed on the patient is finished.

If one medical treatment is finished (YES at step S16), the medical information processing apparatus 6 calculates the attribute information of the medical treatment (step S18). Specifically, the attribute information calculation function 21 of the processing circuitry 15 calculates the attribute information of the medical treatment. In the example of FIG. 6, for example, the proficiency score of the medical technician who performed the catheter treatment is calculated based on the medical procedure record data D10 managed by the medical information manager 3 and the medical data memory 4 and set as the attribute information.

Thereafter, as shown in FIG. 5, the medical information processing apparatus 6 has the medical data memory 4 store the attribute information calculated at step S18 as the medical data D3 (step S20). Specifically, the medical data storage function 24 of the processing circuitry 15 has the medical data memory 4 store the calculated attribute information as the ancillary information data D5 that is associated with the entire medical treatment. The medical information generation process in the embodiment is then ended.

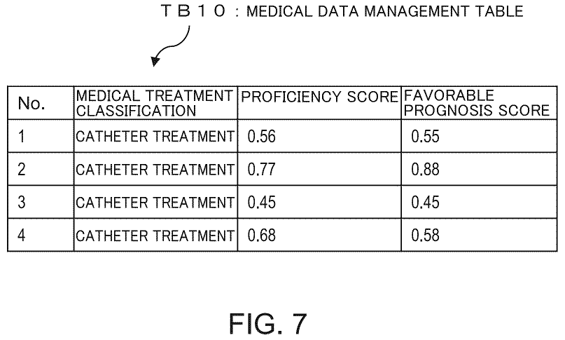

FIG. 7 shows an example of a format of a medical data management table TB10 held in the medical information processing system 1 according to the embodiment. The medical data management table TB10 shown in FIG. 7 is used for managing the medical procedure record data D10 and the medical data D3 stored in the medical data memory 4. In the embodiment, the medical data management table TB10 is stored in the medical information manager 3, for example. However, where the medical data management table TB10 is stored is not limited to the medical information manager 3, but may be the medical data memory 4 or the storage circuity 12 of the medical information processing apparatus 6.

As shown in FIG. 7, the medical data management table TB10 according to the embodiment shows the classification of medical treatment, the proficiency score of the medical technician who have performed the medical treatment, and the favorable prognosis score of the medical treatment. The classification of the medical treatment and the proficiency score can be acquired from the ancillary information data D5 in the medical data D3. The favorable prognosis score is generated by the medical information processing apparatus 6 or the medical information manager 3 after a predetermined period of time passes from a medical treatment by judging the prognosis based on the patient's medical procedure record data D10. Therefore, the favorable prognosis score is added to the medical data management table TB10 after a predetermined period of time passes from the medical treatment.

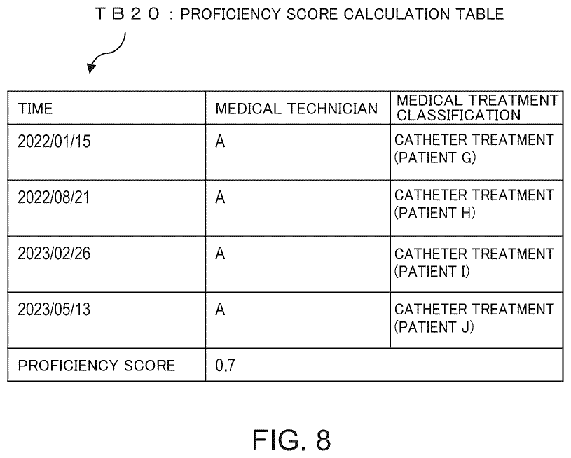

FIG. 8 shows an example of a proficiency score calculation table TB20 for calculating the proficiency score. In the embodiment, the proficiency score calculation table TB20 shown in FIG. 8 is stored in the medical information manager 3, for example. However, where the proficiency score calculation table TB20 is stored is not limited to the medical information manager 3, but may be the medical data memory 4 or the storage circuity 12 of the medical information processing apparatus 6. The proficiency score calculation table TB20 may be temporality generated when the attribute information calculation function 21 of the medical information processing apparatus 6 calculates the proficiency score.

As shown in FIG. 8, the proficiency score calculation table TB20 according to the embodiment has a format including the time when the medical treatment is performed, the name of medical technician who performed the medical treatment, and the classification of the medical treatment. FIG. 8 shows information on the catheter treatments performed by a medical technician A in order to calculate the proficiency score of the catheter treatments performed by the medical technician A. In the example of FIG. 8, the medical technician A has performed the catheter treatments four times, two in the year 2022 and two in the year 2023.

The medical information processing apparatus 6 calculates the proficiency score based on the number of times the medical technician has performed the medical treatments in the past, and the intervals of the medical treatments. For example, the medical information processing apparatus 6 increases the proficiency score as the number of medical treatments in the same classification increases, and decreases the proficiency score if there is a long time interval between medical treatments. Thus, the proficiency score is repeatedly calculated and updated at predetermined time intervals for the same medical technician.

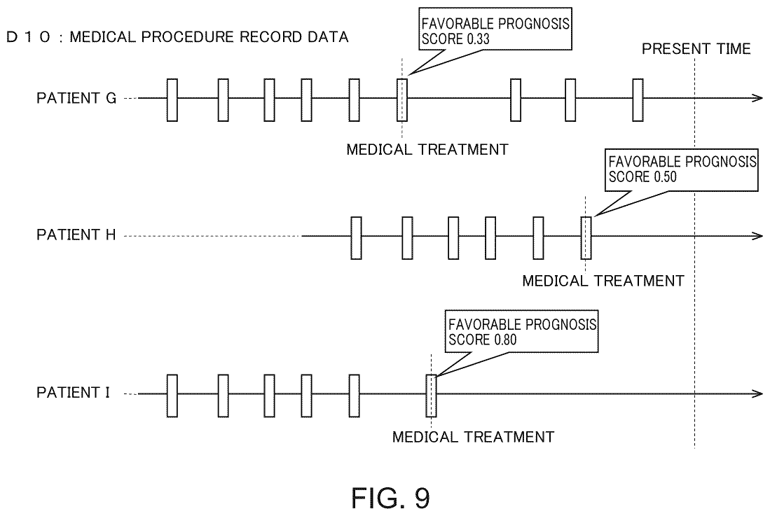

FIG. 9 is a diagram for explaining an example of a logic with which the medical information manager 3 or the medical information processing apparatus 6 calculates the favorable prognosis score. FIG. 9 shows the medical procedure record data D10 of each of the patient G, the patient H, and the patient I. The patient G received a medical treatment a long time ago from now and has received certain medical treatments since then. Therefore, it cannot be said that the prognosis of the patient G is favorable. The favorable prognosis score is thus calculated as 0.33.

The patient I also received a medical treatment a long time ago from now, and has received no medical treatment since then. It can therefore be said that the prognosis is more favorable for the patient I than the patient G. The favorable prognosis score is therefore calculated as 0.80.

Not much time has passed since the patient H received a medical treatment until now. Thus, the period of time from the medical treatment to now is shorter for the patient H than for the patient G or the patient I. The favorable prognosis score of the patient H is therefore calculated as 0.50. The favorable prognosis score relating to the medical treatments performed on the patient H may become higher if a long period of time passes from the last medical treatment. If a further medical treatment is performed on the patient H, the favorable prognosis score will be lowered.

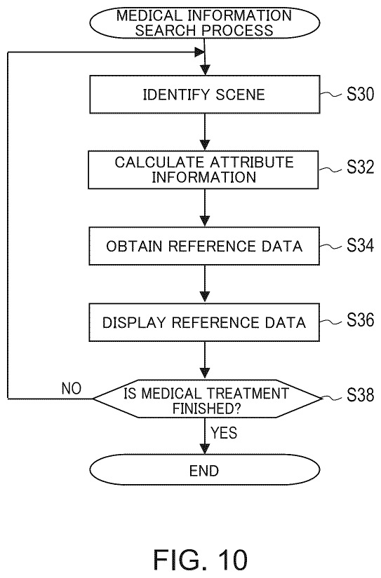

Next, a medical information search process performed by the medical information processing apparatus 6 according to the embodiment will be described with reference to FIG. 10. The medical information search process is performed by the processing circuitry 15 by reading and executing a medical information search process program stored in the storage circuity 12 of the medical information processing apparatus 6. The medical information search process may also be performed when a medical technician or a clinical examination technician, for example, inputs an instruction to start the medical treatment to the medical information processing apparatus 6. Thus, when a medical treatment is started, the medical information processing apparatus 6 according to the embodiment simultaneously performs the medical information search process and the above-described medical information generation process shown in FIG. 5.

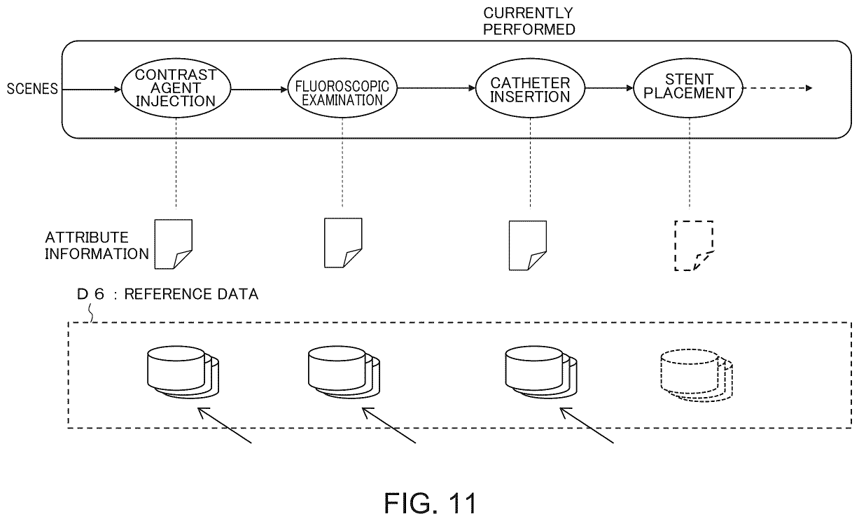

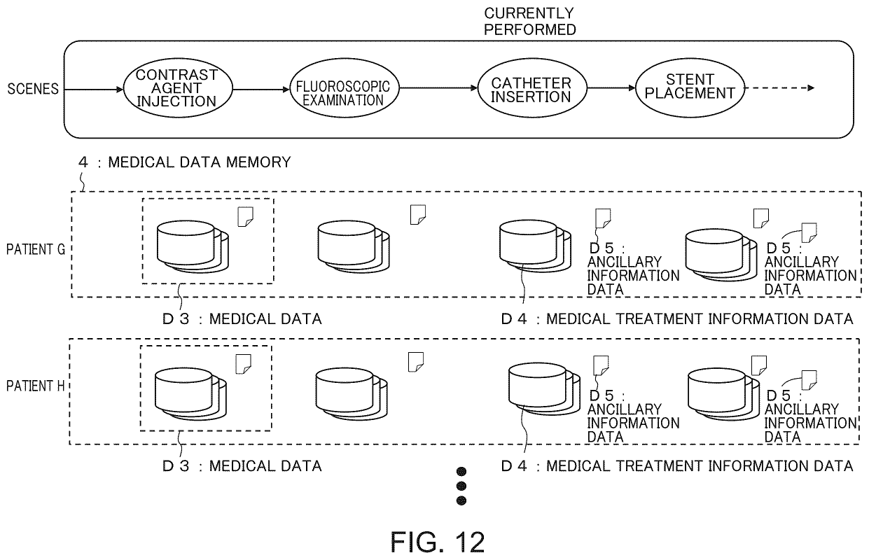

FIGS. 11 and 12 are diagrams for explaining the flow of data in the medical information search process when a catheter treatment is performed as the medical treatment on the patient F, like the above-described medical information generation process shown in FIG. 5. The medical information search process shown in FIG. 10 will be described taking a catheter treatment performed as the medical treatment on the patient F as an example, with reference to FIGS. 11 and 12.

In the medical information search process shown in FIG. 10, first the medical information processing apparatus 6 identifies a scene in the medical treatment performed on the patient (step S30). Specifically, like step S10 described above, the scene identification function 20 of the processing circuitry 15 identifies a scene in the medical treatment performed on the patient.

As in the descriptions with reference to FIG. 6, in the example of FIGS. 11 and 12, the scene identification function 20 performs image analysis and identifies that the scene performed on the patient as a medical treatment is a catheter insertion scene included in the catheter treatment. When identifying the scene, the scene identification function 20 may take the sequential order of a plurality of scenes into account. For example, a scene that comes after the contrast agent injection and the fluoroscope examination scene may be determined as the catheter insertion scene if the treatment proceeds without a problem.

Next, as shown in FIG. 10, the medical information processing apparatus 6 calculates attribute information relating to the attribute of the scene identified in step S30 (step S32). Specifically, like step S12 described above, the attribute information calculation function 21 of the processing circuitry 15 calculates the attribute information relating to the attribute of the scene identified by the scene identification function 20. The attribute information calculated by the attribute information calculation function 21 at step S32 may be either the same as or different from the attribute information calculated by the attribute information calculation function 21 at step S12 described above. Since the attribute information calculated at step S32 is used for retrieving reference data for the scene, the attribute information may have less items than the attribute information calculated at step S12 in order to narrow the search scope. On the contrary, the number of items in the attribute information calculated at step S32 may be more than the number of items of the attribute information calculated at step S12 to broaden the search scope.

Next, as shown in FIG. 10, based on the attribute information calculated at step S32, the medical information processing apparatus 6 searches the medical database, in which the past medical data is stored, for reference data that matches the scene identified at step S30 (step S34). Specifically, the reference data acquisition function 22 of the processing circuitry 15 searches the medical database based on the attribute information calculated by the attribute information calculation function 21 to obtain reference data D6 that matches the scene identified by the scene identification function 20.

As described above, in the embodiment, the medical procedure record data D10 of a plurality of patients is stored in the medical data memory 4 to constitute the medical database. The reference data acquisition function 22 searches the medical database formed in the medical data memory 4 to obtain the reference data that matches the scene identified at step S30.

In the embodiment, the medical data D3 of the medical procedure record data D10 in the medical database includes the medical treatment information data D4 of the patient and the ancillary information data D5 associated with the medical treatment information data D4. The reference data acquisition function 22 therefore searches for the ancillary information data D5 in the medical database that matches the attribute information calculated by the attribute information calculation function 21. The reference data acquisition function 22 then acquires the medical treatment information data D4 associated with the ancillary information data D5 that matches the attribute information as the reference data D6. In the embodiment, the medical treatment information data D4 is image data taken for the same scene. The reference data acquisition function 22 therefore acquires the image data of another patient that is the closest to the identified scene as the reference data D6.

In the example of FIGS. 11 and 12, the medical database, in which data of catheter treatments performed on a plurality of patients in the past is accumulated, is formed in the medical data memory 4. The reference data acquisition function 22 identifies a piece of the ancillary information data D5 that is the closest to the calculated attribute information by performing a character search in the ancillary information data D5. The reference data acquisition function 22 then acquires a piece of the image data, which is a piece of the medical treatment information data D4 associated with the closest piece of ancillary information data D5, as the reference data D6.

As described above, if the ancillary information data D5 includes the proficiency score of the medical technician as proficiency information, the reference data acquisition function 22 may use the proficiency information to acquire the reference data that matches the identified scene. If a plurality of pieces of ancillary information data D5 are found to match the attribute information, the reference data acquisition function 22 may acquire a piece of medical treatment information data D4 that is associated with a piece of ancillary information data D5 having the highest proficiency score as the reference data D6.

If the ancillary information data D5 includes a favorable prognosis score that indicates a favorable prognosis of the medical treatment, the reference data acquisition function 22 may acquire the reference data that matches the identified scene using the favorable prognosis score. For example, if a plurality of pieces of ancillary information data D5 are found to match the attribute information, the reference data acquisition function 22 may acquire a piece of medical treatment information data D4 that is associated with a piece of ancillary information data D5 having the highest favorable prognosis score as the reference data D6.

Next, as shown in FIG. 10, the medical information processing apparatus 6 causes the display to display the reference data D6 acquired at step S34 (step S36). Specifically, the reference data display function 23 of the processing circuitry 15 causes the display 10 to display the reference data D6 acquired by the reference data acquisition function 22. The display used by the reference data display function 23 to display the reference data D6 is not limited to the display 10 of the medical information processing apparatus 6 but may be a display at an arbitrarily determined location. For example, the reference data display function 23 may cause the display of the controller 5 to display the reference data D6, or to cause the display of the medical data generator 2 to display the reference data D6.

Since the reference data D6 is displayed on a display, the user, who is the medical technician, may check in real time a piece of reference data D6 of a past scene that is in a similar situation to a current situation, and may perform the medical treatment on the patient more accurately. Thus, checking the past reference data D6 helps compensate for insufficient experience of the user, who is the medical technician.

In the embodiment, the reference data D6 may be displayed to be arranged with the image data of the medical treatment that is currently performed by the user, the medical technician. The image data corresponds to the image data used for identifying the scene at step S30. By arranging the two pieces of image data side by side, the user or the medical technician may observe and compare the two pieces of image data easily.

In the embodiment, the scene reference data D6 may be displayed together with the ancillary information data D5 associated with the medical treatment information data D4 corresponding to the reference data D6. As a result, the user or the medical technician may also use the ancillary information data D5, which is character information, as reference information.

As described above, the medical information processing apparatus 6 and the medical information processing system 1 including the medical information processing apparatus 6 according to the embodiment is able to retrieve the reference data D6 that matches a scene of a currently performed medical treatment from the medical database including the medical procedure record data D10 of a vast number of patients. Therefore, the medical technician or the clinical examination technician, who is the user, may be able to easily refer to a piece of medical treatment information data D4 that corresponds to the closest scene. Furthermore, since no preparation is needed to provide in real time the reference data D6 that is suitable for a changing state or situation, it is possible to supply appropriate support information that helps compensate for insufficient experience of the medical technician, for example, during a medical treatment.

The term “processor” herein means circuitry such as a central processing unit (CPU), a graphics processing unit (GPU), an application specific integrated circuit (ASIC), a programmable logic device (for example, a simple programmable logic device (SPLD) or a complex programmable logic device (CPLD)), or a field programmable gate array (FPGA). The processor performs a function by reading and executing a program stored in the storage circuitry. Instead of being stored in the storage circuity, a program may be directly integrated into the circuitry of the processor. In such a case, the processor performs the function by reading and the executing the program integrated into the circuitry. The processor may be formed of a single circuit, or a combination of a plurality of distinct circuits to perform the function. A plurality of components may be integrated into a single processor to perform their respective functions.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. A medical information processing apparatus comprising:
a display; and
processing circuitry configured to
identify a scene in a medical treatment performed on a patient as an identified scene,
calculate attribute information relating to an attribute of the identified scene,
search a medical database to acquire a piece of reference data that matches the identified scene based on the attribute information, wherein medical data generated in the past is accumulated in the medical database, and
cause the display to display the piece of the reference data.

2. The medical information processing apparatus according to claim 1, wherein the medical data accumulated in the medical database comprises:
medical treatment information data of the patient, wherein the medical treatment information data is associated with each scene; and
ancillary information data, which is information associated with the medical treatment information data.

3. The medical information processing apparatus according to claim 2, wherein the processing circuitry is further configured to, when acquiring the piece of the reference data, search through the ancillary information data in the medical database based on the attribute information, and acquires a piece of the medical treatment information data corresponding to a piece of the ancillary information data that matches the attribute information as the reference data.

4. The medical information processing apparatus according to claim 2, wherein in the medical database, the medical treatment and a plurality of scenes included in the medical treatment are associated with each other.

5. The medical information processing apparatus according to claim 2, wherein the medical database stores proficiency information as the ancillary information data, wherein the proficiency information relates to a degree of proficiency of a medical technician who performed the medical treatment on the patient.

6. The medical information processing apparatus according to claim 5, wherein the processing circuitry is further configured to acquire the piece of the reference data that matches the identified scene using the proficiency information included in the ancillary information data.

7. The medical information processing apparatus according to claim 2, wherein the medical database comprises a favorable prognosis score as the ancillary information data, wherein the favorable prognosis score indicates a favorable prognosis tendency after the medical treatment is performed.

8. The medical information processing apparatus according to claim 7, wherein the processing circuitry is further configured to acquire the piece of the reference data that matches the identified scene using the favorable prognosis score included in the ancillary information data.

9. The medical information processing apparatus according to claim 1, wherein the processing circuitry is further configured to identify the identified scene based on an image of the medical treatment performed on the patient.

10. The medical information processing apparatus according to claim 9, wherein the processing circuitry is further configured to cause the display to display the piece of the reference data and the image side by side on the display.

11. A medical information processing apparatus comprising:
a medical data memory; and
processing circuitry configured to
identify a scene in a medical treatment performed on a patient as an identified scene,
calculate attribute information relating to an attribute of the identified scene, and
store medical data in the medical data memory, wherein the medical data includes medical treatment information data which corresponds to the patient and which is associated with the attribute information.

12. A medical information processing apparatus comprising:
processing circuitry configured to
acquire situation information which indicates a situation of medical treatment performed on a patient,
acquire reference information based on the situation information, wherein the reference information is associated with the situation of the medical treatment, and
output the reference information.

13. The medical information processing apparatus according to claim 12, further comprising a memory in which the situation information and the reference information are stored so as to be associated with each other,
wherein the processing circuitry is further configured to acquire, from the memory, the reference information associated with the situation information.

14. The medical information processing apparatus according to claim 13, wherein the processing circuitry is further configured to
acquire a list of the reference information from the memory based on the medical treatment before the situation information is acquired, and
acquire the reference information associated with the situation information by using the list acquired previously.

15. The medical information processing apparatus according to claim 12, wherein the situation information includes a degree of proficiency of a medical technician, and
the processing circuitry is further configured to acquire the reference information associated with the reference information including a high degree of proficiency.

16. The medical information processing apparatus according to claim 12, wherein the situation information includes a degree of proficiency of a medical technician, and
the processing circuitry is further configured to acquire the reference information associated with the degree of proficiency.

17. The medical information processing apparatus according to claim 12, wherein the situation information includes a favorable prognosis tendency after the medical treatment is performed, and
the processing circuitry is further configured to acquire the reference information associated with a high favorable prognosis.

18. The medical information processing apparatus according to claim 17, wherein the favorable prognosis includes the medical treatment performed on the patient and a favorable prognosis score indicating a favorable prognosis tendency after the medical treatment is performed.

19. The medical information processing apparatus according to claim 12, wherein the processing circuitry is further configured to cause a display to display an image taken for a scene in the medical treatment and the reference information.

20. The medical information processing apparatus according to claim 12, wherein the situation information indicates, as a feature amount of the medical treatment, a situation identified by analyzing an image taking for a scene in the medical treatment and a situation identified by analyzing a scene in the medical treatment performed on the patient.
